(19) United States
(12) Patent Application Publication
(10) Pub. No.: US 2014/0135699 A1
(43) Pub. Date: May 15, 2014

(54) SYSTEMS AND METHODS FOR DELIVERING A THERAPEUTIC AGENT

(71) Applicant: J. Richard Gyory, Sudbury, MA (US)

(72) Inventor: J. Richard Gyory, Sudbury, MA (US)

(73) Assignee: SpringLeaf Therapeutics, Inc., Boston, MA (US)

(21) Appl. No.: 13/673,464

(22) Filed: Nov. 9, 2012

(51) Int. Cl. A61M 5/145

(52) U.S. Cl. CPC A61M 5/145
USPC 604/134; 604/151

(57) ABSTRACT

A delivery system includes a reservoir configured to contain a fluid and a fluid communicator configured to be placed in fluid communication with the reservoir. A first actuator is coupled to the reservoir and configured to exert a first force on the reservoir upon actuation. A transfer structure disposed between the first actuator and the reservoir is configured to engage the reservoir distribute the first force across a surface of the reservoir. A second actuator is disposed and oriented so that when the first actuator is displaced toward the fluid reservoir, the second actuator moves from a first configuration to a second configuration and exerts a second force on the transfer structure. The combination of the first and second forces collectively urge the transfer structure towards the reservoir such that fluid within the reservoir is communicated through the fluid communicator.

### CROSS-REFERENCE TO RELATED APPLICATIONS

This application is related to U.S. Patent Publication No. 2011/0275997, entitled “Systems and Methods for Delivering a Therapeutic Agent,” filed on May 5, 2011, and U.S. Patent Publication No. 2011/0275998, entitled “Systems and Methods for Delivering a Therapeutic Agent,” filed on May 6, 2011, the disclosures of which are incorporated herein by reference in their entirety.

### BACKGROUND

Embodiments described herein relate generally to medical devices and procedures, including, for example, medical devices and methods for delivering a therapeutic agent to a patient.

Drug delivery involves delivering a drug or other therapeutic compound into the body. Typically, the drug is delivered via a technology that is carefully selected based on a number of factors. These factors can include, but are not limited to, the characteristics of the drug, such as drug dose, pharmacokinetics, complexity, cost, and absorption, the characteristics of the desired drug delivery profile (such as uniform, non-uniform, or patient-controlled), the characteristics of the administration mode (such as the ease, cost, complexity, and effectiveness of the administration mode for the patient, physician, nurse, or other caregiver), or other factors or combinations of these factors.

Conventional drug delivery technologies present various challenges. Oral administration of a dosage form is a relatively simple delivery mode, but some drugs may not achieve the desired bioavailability and/or may cause undesirable side effects if administered orally. Further, the delay from time of administration to time of efficacy associated with oral delivery may be undesirable depending on the therapeutic need. While parenteral administration by injection may avoid some of the problems associated with oral administration, such as providing relatively quick delivery of the drug to the desired location, conventional injections may be inconvenient, difficult to self-administer, and painful or unpleasant for the patient. Furthermore, injection may not be suitable for achieving certain delivery/release profiles, particularly over a sustained period of time.

Passive transdermal technology, such as a conventional transdermal patch, may be relatively convenient for the user and may permit relatively uniform drug release over time. However, some drugs, such as highly charged or polar drugs, peptides, proteins and other large molecule active agents, may not penetrate the stratum corneum for effective delivery. Furthermore, a relatively long start-up time may be required before the drug takes effect. Thereafter, the drug release may be relatively continuous, which may be undesirable in some cases. Also, a substantial portion of the drug payload may be undeliverable and may remain in the patch once the patch is removed.

Active transdermal systems, including iontophoresis, sonophoresis, and poration technology, may be expensive and may yield unpredictable results. Only some drug formulations, such as aqueous stable compounds, may be suited for active transdermal delivery. Further, modulating or controlling the delivery of drugs using such systems may not be possible without using complex systems.

Some infusion pump systems may be large and may require tubing between the pump and the infusion set, which can impact the quality of life of the patient. Further, infusion pumps may be expensive and may not be disposable. From the above, it would be desirable to provide new and improved drug delivery systems and methods that overcome some or all of these and other drawbacks.

### SUMMARY OF THE INVENTION

Devices and methods for delivering a fluid to a patient are disclosed herein. In one embodiment, a delivery system includes a reservoir configured to contain a fluid and a fluid communicator configured to be placed in fluid communication with the reservoir. A first actuator is coupled to the reservoir and configured to exert a first force on the reservoir upon actuation such that fluid within the reservoir is communicated through the fluid communicator. The first actuator includes a first end that is constrained and a second end that is not constrained. The first actuator is configured to bend at a location along a length of the actuator when actuated such that the second end of the actuator is displaced in a direction toward the fluid reservoir. The first actuator can be an electrochemical actuator. The apparatus further includes a transfer structure disposed between the first actuator and the reservoir configured to engage the reservoir such that the first force exerted by the first actuator is distributed by the transfer structure across a surface of the reservoir engaged by the transfer structure. A second actuator is disposed and oriented so that when the second end of the first actuator is displaced toward the fluid reservoir, the second actuator moves from a first configuration to a second configuration and exerts a second force on the transfer structure. The combination of the first force and the second force collectively urge the transfer structure towards the reservoir such that fluid within the reservoir is communicated through the fluid communicator.

### BRIEF DESCRIPTION OF THE DRAWINGS

FIG. 1 is a schematic illustration of a fluid delivery system according to an embodiment.

FIG. 2 is a perspective view of a fluid delivery system according to an embodiment.

FIG. 3 is an exploded view of the delivery system of FIG. 2.

FIG. 4 is an exploded view of an actuator sub-assembly included in the delivery system of FIG. 2.

FIG. 5 is an exploded view of a mechanical actuator included in the delivery system of FIG. 2.

FIG. 6A-6C are perspective views of a fluid delivery system according to an embodiment in a first, second and third configuration, respectively.

FIG. 7A-7C are perspective views of a fluid delivery system according to an embodiment in a first, second and third configuration, respectively.

FIG. 8A-8C are cross sectional views of the actuator sub-assembly of FIG. 3 taken along the line A-A in the first, second and third configuration, respectively.

FIG. 9A-9C are top views of the actuator sub-assembly of FIG. 3 in a first, second and third configuration, respectively.

### DETAILED DESCRIPTION

Devices, systems and methods are described herein that are configured for use in the delivery of therapeutic agents to a patient's body. Such therapeutic agents can be, for example, one or more drugs and can be in fluid form of various viscosities. In some embodiments, the devices and methods can include a pump device that includes an actuator, such as, for example, an electrochemical actuator, which can have characteristics of both a battery and a pump. Specifically, an electrochemical actuator can include an electrochemical cell that produces a pumping force as the cell discharges. Thus, the pump device can have relatively fewer parts than a conventional drug pump, such that the pump device is relatively more compact, disposable, and reliable than conventional drug pumps. Such drug delivery devices are desirable, for example, for use in delivery devices that are designed to be attached to a patient's body (e.g., a wearable device). These attributes of the pump device may reduce the cost and the discomfort associated with infusion drug therapy.

In some embodiments, such a pump device can be operated with, for example, a controller and/or other circuitry, operative to regulate drug or fluid flow from the pump device. Such a controller may permit implementing one or more release profiles using the pump device, including release profiles that require uniform flow, non-uniform flow, continuous flow, discontinuous flow, programmed flow, scheduled flow, user-initiated flow, or feedback responsive flow, among others. Thus, the pump device may effectively deliver a wider variety of drug therapies than other pump devices.

The systems and methods described herein can include an electrochemical actuator, such as a self-powered actuator and/or combined battery and actuator. Example embodiments of such electrochemical actuators are generally described in U.S. Pat. No. 7,541,715, entitled “Electrochemical Methods, Devices, and Structures” by Chiang et al., U.S. Pat. No. 7,872,396, entitled “Electrochemical Actuator” by Chiang et al., U.S. Pat. No. 7,999,436, entitled “Electrochemical Actuator” by Chiang et al., U.S. Pat. No. 7,828,771, entitled “Systems and Methods for Delivering Drugs” by Chiang et al., (the '771 patent), and U.S. Pat. No. 8,247,946, entitled “Electrochemical Actuator” by Chiang et al., (collectively referred to herein as the “the Electrochemical Actuator applications”), the disclosures of which are incorporated herein by reference in their entirety. Such electrochemical actuators can include at least one component that upon discharge from an initially charged state, or upon the application of a voltage or current, responds by experiencing a change in volume or position. The change in volume or position can produce mechanical work that can then act on a fluid source or may be transferred to a fluid communicator, such that a fluid can be delivered out of the fluid source.

In some embodiments of a delivery system, an electrochemical actuator is configured as an elongate plate, which bends when actuated. The actuator can be clamped or otherwise constrained at one end, so that the actuator is cantilevered from that end. In such an arrangement, an increased range of motion at the free end for the same angular deflection of the electrochemical actuator can be achieved and/or an increased rate of actuation for the same vertical tip deflection. In some cases, an increased range of motion and/or an increased actuation rate can also result in a reduction in the tip force that it can apply to pump fluid out of a fluid reservoir. This change in force may be dependent on the externally applied load that affects the stress at the clamp location. In some embodiments, however, such a reduction in tip force can be tolerated. In some embodiments, a delivery device having an electrochemical actuator with one end constrained, can result in approximately doubling of the vertical deflection of the actuator. Thus, the useful stroke of the actuator can be effectively doubled (depending on angular actuator displacement). In general, the size of the actuator, coupled with the location of the push (e.g., location where the actuator pushes on a transfer structure and/or fluid source as described herein) and cantilever points can be leveraged to change the interplay of vertical displacement and available force. With the same actuator, moving the push point closer to the pivot will act to increase the piston's displacement rate but also increase the force requirements on the actuator. Conversely, moving the push point away from the pivot will act to decrease force requirements (thus enabling the use of weaker, possibly cheaper actuators) but also reduce displacement rate, and require generally larger vertical stroke from the actuator.

Electrochemical actuators can provide volume-efficient capabilities that are especially effective in applications where minimal weight and volume are desired. Example applications are those of drug/medication patch pumps that are worn by a patient. While most pumps use a variety of prime movers that either require external drive circuitry or power, or are bulky, expensive, and/or complex, electrochemical actuator-based pumps have significant advantages by virtue of having a small actuator volume and no need for an external power source.

By clamping an end of an electrochemical actuator used in a drug delivery device, the device and/or actuator can be asymmetric, thus further saving both volume and material (and therefore cost). In some embodiments of a drug delivery device, an electrochemical actuator can include rigid external legs coupled to one end or opposite ends of the actuator. A rigid leg can be used as an interface between the actuator and the clamping mechanism and can also house suitable drive electronics (from the simplest version of a discharge resistor and an activation switch to more complex communication units). This additional configuration can further optimize features of the basic electrochemical actuator (such as minimal size that can sustain the load, reduced complexity and cost, simple fabrication, etc.) and leave interfacing with loads and the package to the external legs. The electronics can include some or all of the necessary drive circuitry, communication units, as well as a switch to activate motion as needed.

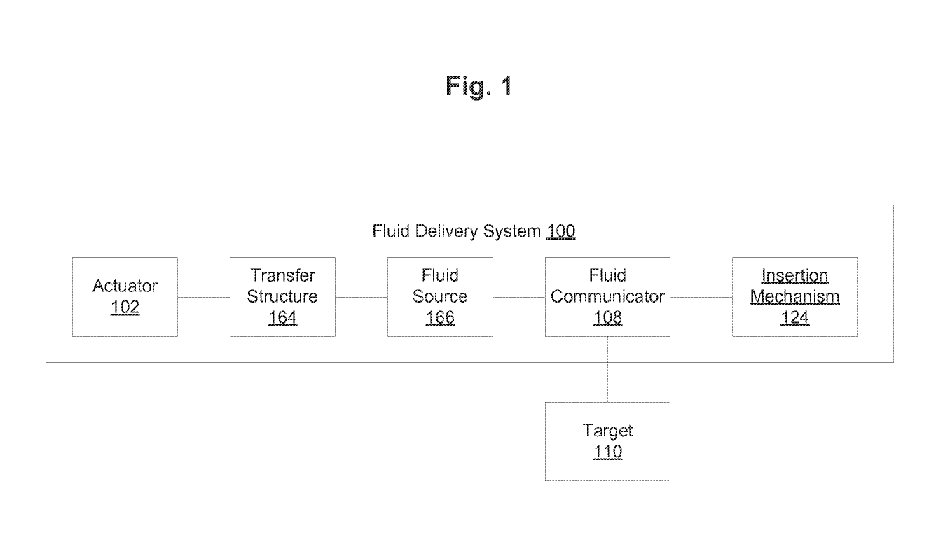

FIG. 1 is a schematic block diagram illustrating an embodiment of a fluid delivery system 100 (also referred to herein as “delivery device” or “drug delivery device”). The fluid delivery system 100 includes an actuator 102, a transfer structure 164, a fluid source 166, a fluid communicator 108 and an insertion mechanism 124. The fluid source 166 can contain a fluid (i.e., a therapeutic agent) to be delivered into a target 110 via the fluid communicator 108. The target 110 can be, for example, a human or other mammalian body in need of a drug therapy or prophylaxis.

The actuator 102 can include, for example, an electrochemical actuator that can actuate or otherwise create a pumping force to deliver the fluid from the fluid source 166 into the fluid communicator 108. In some embodiments, the actuator 102 can be a device that experiences a change in volume or position in response to an electrochemical reaction that occurs therein. For example, the actuator 102 can be an electrochemical actuator that includes a charged electrochemical cell, and at least a portion of the electrochemical cell can actuate as the electrochemical cell discharges as described in the Electrochemical Actuator applications incorporated by reference above. Thus, the electrochemical actuator can be considered a self-powered actuator or a combination battery and actuator.

In some embodiments, the electrochemical actuator can include a positive electrode and a negative electrode, at least one of which is an actuating electrode. These and other components of the electrochemical actuator can form an electrochemical cell, which in some embodiments can initially be charged. For example, the electrochemical cell may begin discharging when a circuit between the electrodes is closed, causing the actuating electrode to actuate. The actuating electrode can thereby perform work upon another structure such as the fluid source 166, or a transfer structure 164 associated with the fluid source 166 as described in more detail below. The work can then cause fluid to be pumped or otherwise dispensed from the fluid source 166 into the target 110.

More specifically, the actuating electrode of the electrochemical actuator can experience a change in volume or position when the closed circuit is formed, and this change in volume or position can perform work upon the fluid source 166 or transfer structure 164. For example, the actuating electrode may expand, bend, buckle, fold, cup, elongate, contract, or otherwise experience a change in volume, size, shape, orientation, arrangement, or location, such that at least a portion of the actuating electrode experiences a change in volume or position. In some embodiments, the change in volume or position may be experienced by a portion of the actuating electrode, while the actuating electrode as a whole may experience a contrary change or no change whatsoever. It is noted that the delivery device 100 can include more than one electrochemical actuator. For example, in some embodiments, the delivery device 100 can include one or more electrochemical actuators arranged in series, parallel, or some combination thereof. In some embodiments, a number of such electrochemical actuators may be stacked together. As another example, concurrent or sequenced delivery of multiple agents can be achieved by including one or more electrochemical actuators acting on two or more fluid sources.

In some embodiments, the electrochemical actuator can be constrained (also referred to herein as “fixed”), at one end, e.g. by coupling to a clamping mechanism (not shown in FIG. 1) and the opposite end can be unconstrained. With one end constrained, the electrochemical actuator can deflect or bend when activated such that the free end bends or rotates about a bend axis as described in more detail below. The transfer structure 164 can be pivotally coupled at one end to a mounting member (not shown in FIG. 1) and include a free end at an opposite end such that upon activation of the electrochemical actuator, the transfer structure 164 can pivot about its pivot coupling. For example, as the electrochemical actuator is activated and begins to bend or deflect, the actuating electrode can contact and exert a force on the transfer structure 164 and cause the transfer structure 164 to move or rotate about its pivotal coupling. As the transfer structure 164 moves, it can contact the fluid source 166 as described above, to cause the fluid within the fluid source 166 to be discharged out of the fluid source 166 and into the patient. In some embodiments, the electrochemical actuator can be coupled (e.g., clamped) to the transfer structure 164 and configured to push against the bottom of the housing. This may be a desirable configuration to optimize how the system is supplied and/or assembled/activated by the end user. In some embodiments, the electrochemical actuator can be coupled to an intermediate structure so that the force generated by the bending electrode is contained by the intermediate structure and not transmitted to the housing of the delivery system 100.

In some embodiments the actuator 102 can include, for example, a mechanical actuator such as a spring-based actuator, a piezoelectric actuator, a hydraulic actuator or a combination thereof. In some embodiments the mechanical actuator can include a spring-based actuator that can use the displacement produced by a spring or a member attached to the spring to create a pumping force to communicate the fluid from the fluid source 166 into the fluid communicator 108. For example, the mechanical actuator can include a torsion spring coupled to a rotary compression member and can be configured such that when the torsion spring is released from its compressed (or “twisted”) configuration, the rotational motion of the rotary compression member applies a compressive force to the fluid source 166. In some embodiments, the rotary compression member can be configured to convert the rotational motion of the rotary compression member to an axial compressive force on the fluid source 166. For example, the rotary compression member can have an edge or a surface that is shaped to engage the transfer structure 164 and apply a compressive force on the fluid source 166. The shaped surface can be, for example, tapered, curved (e.g., concave or convex) or can include symmetric or asymmetric gradations (e.g., steps with chamfered or filleted edges) or a combination thereof. In some embodiments the rotary compression member can include additional structures such as, for example, a dial-like structure, an arm, a strut, or combination thereof configured to indicate the fluid level in the fluid source 166. In some embodiments, the rotary compression member can be configured to open a circuit between the electrodes of an electrochemical actuator, thereby stopping actuation at the end of the delivery cycle.

In some embodiments the actuator 102 can include multiple actuators (e.g., an electrochemical actuator and a mechanical actuator). For example, the actuator 102 can include an electrochemical actuator configured to apply a first pumping force on the fluid source 166 and a mechanical actuator configured to apply a second pumping force on the fluid source 166. The multiple actuators can be configured to ensure that substantially all of the fluid from the fluid source 166 is delivered to the target 110 via the fluid communicator 108. In some embodiments, two or more mechanical actuators can be used in combination with an electrochemical actuator.

The fluid source 166 can be a reservoir, pouch, chamber, barrel, bladder, or other known device that can contain a drug in fluid form therein. The fluid communicator 108 can be in, or can be moved into, fluid communication with the fluid source 166. The fluid communicator 108 can be, for example, a needle, catheter, cannula, infusion set, or other known drug delivery conduit that can be inserted into or otherwise associated with the target body for drug delivery using the insertion mechanism 124.

In some embodiments, the fluid source 166 can be any component capable of retaining a fluid or drug in fluid form. In some embodiments, the fluid source 166 may be disposable (e.g., not intended to be refillable or reusable). In other embodiments, the fluid source 166 can be refilled, which may permit reusing at least a portion of the device and/or varying the drug or fluid delivered by the device. In some embodiments, the fluid source 166 can be sized to correlate with the electrochemical potential of the electrochemical actuator 102. For example, the size and/or volume of the fluid source 166 can be selected so that the fluid source 166 becomes about substantially empty at about the same time that the electrochemical actuator 102 becomes about substantially discharged. By optimizing the size of the fluid source 166 and the amount of drug contained therein to correspond to the driving potential of the electrochemical actuator 102, the size and/or cost of the device may be reduced. In other embodiments, the electrochemical actuator 102 may be oversized with reference to the fluid source 166. In some embodiments, the delivery system 100 can include more than one fluid source 166. Such a configuration may permit using a single device to deliver two or more drugs or fluids. The two or more drugs or fluids can be delivered discretely, simultaneously, alternating, according to a program or schedule, or in any other suitable manner. In such embodiments, the fluid sources 166 may be associated with the same or different actuators 102, the same or different fluid communicators 108, the same or different operational electronics, or the same or different portions of other components of the delivery system.

The transfer structure 164 can be disposed between the electrochemical actuator 102 and the fluid source 166. The transfer structure 164 includes a surface configured to contact the fluid source 166 upon actuation of the actuator 102 such that a force exerted by the actuator 102 is transferred from the transfer structure 164 to the fluid source 166. The transfer structure 164 can include one or more components. For example, the transfer structure 164 can be a single component having a surface configured to contact the fluid source 166. In some embodiments, the transfer structure 164 can include one or more members having a surface configured to contact the fluid source 166 upon activation of the actuator 102. In some embodiments, the transfer structure 164 is a substantially planar or flat plate.

In some embodiments, the fluid delivery system 100 can be used to deliver a drug formulation which comprises a drug, including an active pharmaceutical ingredient. In other embodiments, the fluid delivery system 100 may deliver a fluid that does not contain a drug. For example, the fluid may be a saline solution or a diagnostic agent, such as a contrast agent. Drug delivery can be subcutaneous, intravenous, intraarterial, intramuscular, intracardiac, intraosseous, intradermal, intrathecal, intraperitoneal, intratumoral, intratympanic, intraaural, topical, epidural, and/or peri-neural depending on, for example, the location of the fluid communicator 108 and/or the entry location of the drug.

The drug (also referred to herein as “a therapeutic agent” or “a prophylactic agent”) can be in a pure form or formulated in a solution, a suspension, or an emulsion, among others, using one or more pharmaceutically acceptable excipients known in the art. For example, a pharmaceutically acceptable vehicle for the drug can be provided, which can be any aqueous or non-aqueous vehicle known in the art. Examples of aqueous vehicles include physiological saline solutions, solutions of sugars such as dextrose or mannitol, and pharmaceutically acceptable buffered solutions, and examples of non-aqueous vehicles include fixed vegetable oils, glycerin, polyethylene glycols, alcohols, and ethyl oleate. The vehicle may further include antibacterial preservatives, antioxidants, tonicity agents, buffers, stabilizers, or other components.

Although the fluid delivery system 100 and other systems and methods described herein are generally described as communicating drugs into a human body, such systems and methods may be employed to deliver any fluid of any suitable biocompatibility or viscosity into any object, living or inanimate. For example, the systems and methods may be employed to deliver other biocompatible fluids into living beings, including human beings and other animals. Further, the systems and methods may deliver drugs or other fluids into living organisms other than human beings, such as animals and plant life. Also, the systems and methods may deliver any fluids into any target, living or inanimate.

The delivery system 100 can also include a housing (not shown in FIG. 1) that can be removably or releasably attached to the body (e.g., the skin) of the patient. The various components of the delivery system 100 can be fixedly or releasably coupled to the housing. For example, the clamping mechanism and the mounting member described above can be formed integrally with a portion of the housing, or can be coupled to the housing.

To adhere the delivery device 100 to the skin of a patient, a releasable adhesive can at least partially coat an underside of the housing. The adhesive can be non-toxic, biocompatible, and releasable from human skin. To protect the adhesive until the device is ready for use, a removable protective covering can cover the adhesive, in which case the covering can be removed before the device is applied to the skin. Alternatively, the adhesive can be heat or pressure sensitive, in which case the adhesive can be activated once the device is applied to the skin. Example adhesives include, but are not limited to, acrylate based medical adhesives of the type commonly used to affix medical devices such as bandages to skin. However, the adhesive is not necessary, and may be omitted, in which case the housing can be associated with the skin, or generally with the body, in any other manner. For example, a strap or band can be used.

The housing can be formed from a material that is relatively lightweight and flexible, yet sturdy. The housing also can be formed from a combination of materials such as to provide specific portions that are rigid and specific portions that are flexible. Example materials include plastic and rubber materials, such as polystyrene, polybutene, carbonate, urethane rubbers, butene rubbers, silicone, and other comparable materials and mixtures thereof, or a combination of these materials or any other suitable material can be used.

In some embodiments, the housing can include a single component or multiple components. In some embodiments, the housing can include two portions: a base portion and a movable portion. The base portion can be suited for attaching to the skin. For example, the base portion can be relatively flexible. An adhesive can be deposited on an underside of the base portion, which can be relatively flat or shaped to conform to the shape of a particular body part or area. The movable portion can be sized and shaped for association with the base portion. In some embodiments, the two portions can be designed to lock together, such as via a locking mechanism. In some cases, the two portions can releasably lock together, such as via a releasable locking mechanism, so that the movable portion can be removably associated with the base portion. To assemble such a housing, the movable portion can be movable with reference to the base portion between an unassembled position and an assembled position. In the assembled position, the two portions can form a device having an outer shape suited for concealing the device under clothing. In some embodiments the base portion can include an insertion mechanism 124 that can house, springs, struts cannulas, needles, activation mechanism, and other structures as necessary that can be used in combination to insert or associate with a needle, catheter, cannula, infusion set, or other fluid delivery conduit into the target 110 for fluid delivery. In some embodiments the fluid communicator 108 can be substantially housed inside the insertion mechanism 124. Various example embodiments of a housing are described in the '771 patent incorporated by reference above.

The size, shape, and weight of the delivery device 100 can be selected so that the delivery device 100 can be comfortably worn on the skin after the device is applied via the adhesive. For example, the delivery device 100 can have a size, for example, in the range of about 1.0″×1.0″×0.1″ to about 5.0″×5.0″×1.0″, and in some embodiments in a range of about 2.0″×2.0″×0.25″ to about 4.0″×4.0″×0.67″. The weight of the delivery device 100 can be, for example, in the range of about 5 g to about 200 g, and in some embodiments in a range of about 15 g to about 100 g. The delivery device 100 can be configured to dispense a volume in the range of about 0.1 ml to about 1,000 ml, and in some cases in the range of about 0.3 ml to about 100 ml, such as between about 0.5 ml and about 5 ml. The shape of the delivery device can be selected so that the delivery device 100 can be relatively imperceptible under clothing. For example, the housing can be relatively smooth and free from sharp edges. However, other sizes, shapes, and/or weights are possible.

As mentioned above, an electrochemical actuator 102 can be used to cause the fluid delivery device 100 to deliver a drug-containing or non-drug containing fluid into a human patient or other target 110. Such a fluid delivery system 100 can be embodied in a relatively small, self-contained, and disposable device, such as a patch device that can be removably attached to the skin of patient as described above. The delivery device 100 can be relatively small and self-contained, in part, because the electrochemical actuator 102 serves as both the battery and a pump. The small and self-contained nature of the delivery device 100 advantageously may permit concealing the device beneath clothing and may allow the patient to continue normal activity as the drug is delivered. Unlike conventional drug pumps, external tubing to communicate fluid from the fluid reservoir into the body can be eliminated. Such tubing can instead be contained within the delivery device 100, and a needle or other fluid communicator 108 can extend from the delivery device 100 into the body. The electrochemical actuator 102 can initially be charged, and can begin discharging once the delivery device 100 is activated to pump or otherwise deliver the drug or other fluid into the target 110. Once the electrochemical actuator 102 has completely discharged or the fluid source 166 (e.g. reservoir) is empty, the delivery device 100 can be removed. The small and inexpensive nature of the electrochemical actuator 102 and other components of the device may, in some embodiments, permit disposing of the entire delivery device 100 after a single use. The delivery device 100 can permit drug delivery, such as subcutaneous or intravenous drug delivery, over a time period that can vary from several minutes to several days. Subsequently, the delivery device 100 can be removed from the body and discarded.

In use, the delivery device 100 can be placed in contact with the target 110 (e.g. placed on the surface of a patient's body), such that the fluid communicator 108 (e.g., a needle, cannula, etc.) is disposed adjacent to a desired injection site. The fluid communicator 108 can be actuated with the actuation of the electrochemical actuator 102 or separately. For example, the delivery device 100 can include a separate mechanism to actuate the fluid communicator 108. Activation of the fluid communicator 108 can include, for example, insertion of the fluid communicator 108 into the patient's body. Example embodiments illustrating various configurations for actuation of the fluid communicator 108 are described in the '771 patent incorporated by reference above. The electrochemical actuator 102 can then be actuated to apply a force on the fluid source 166, causing the fluid to be delivered through the fluid communicator 108 and into the target 110. For example, as the electrochemical actuator 102 is actuated, the actuator 102 will be displaced and will contact and apply a force to the transfer structure 164 and that force will in turn be transferred to the fluid source 166 to pump the fluid out of the fluid source 166, through the fluid communicator 108, and into the target 110.

Having described above various general principles, several exemplary embodiments of these concepts are now described. These embodiments are only examples, and many other configurations of a delivery system and/or the various components of a delivery system, are contemplated.

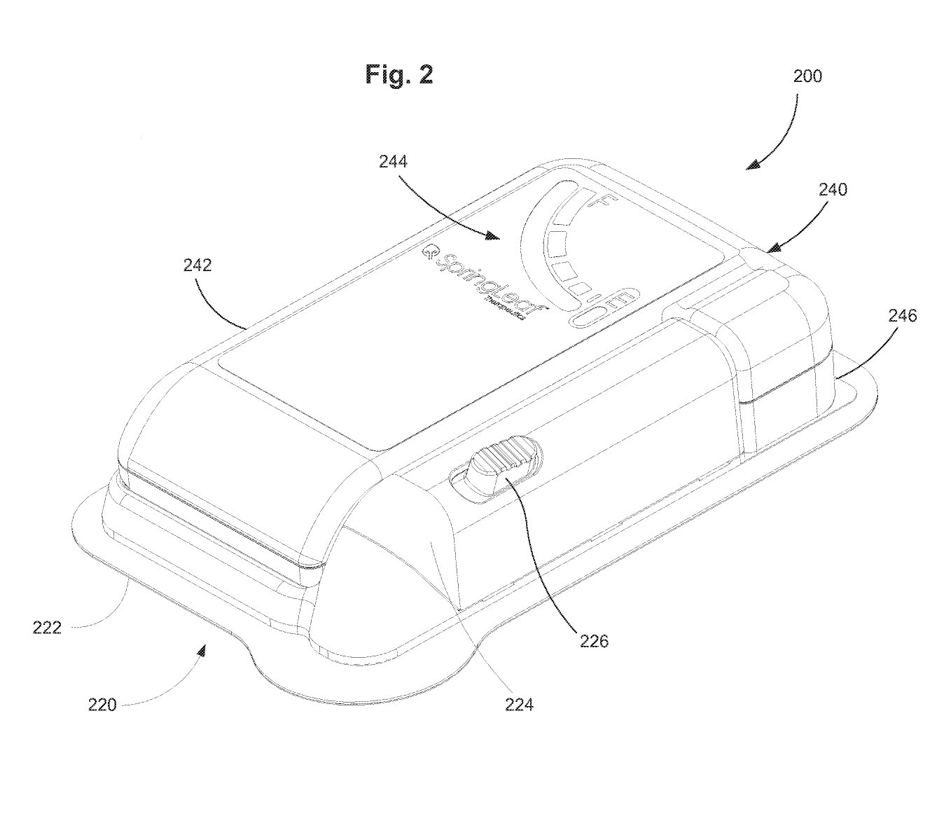

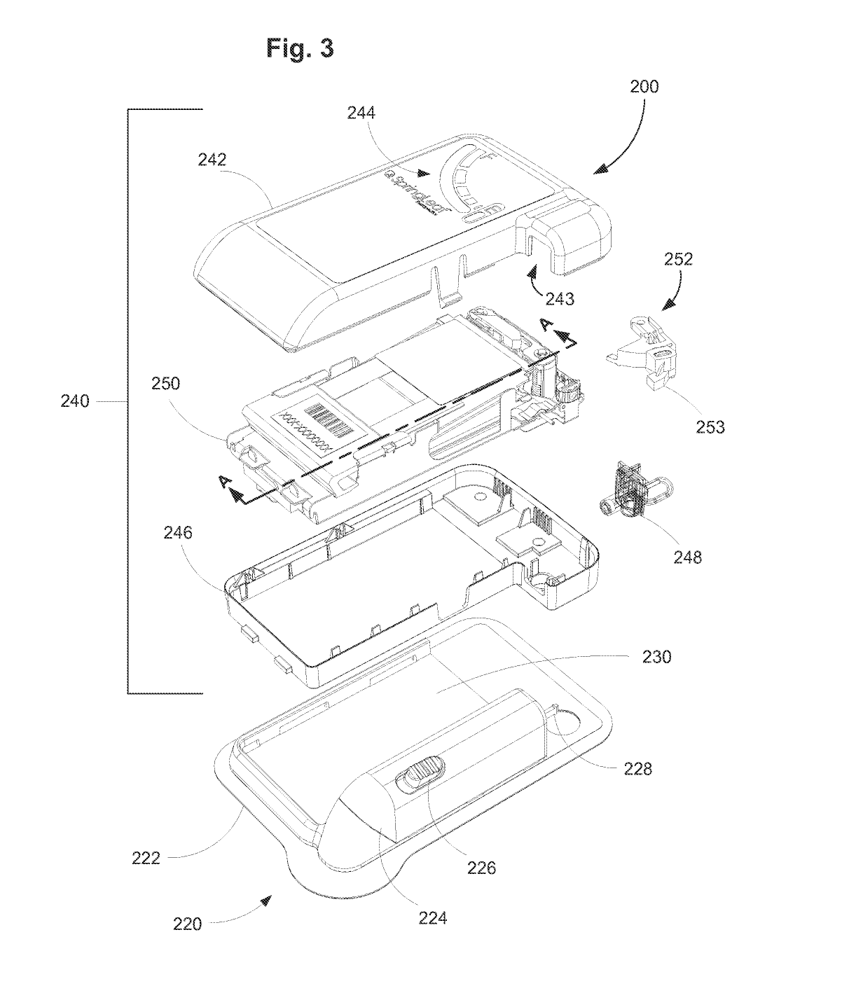

Referring now to FIGS. 2 and 3, a delivery device 200 includes a base portion 220 and a fluid delivery cartridge 240. The base portion 220 and the fluid delivery cartridge 240 can be pre-assembled and permanently coupled together, removably coupled to each other, or separate components that are assembled by the user. The fluid delivery cartridge 240 and the base portion 220 can be coupled together in a similar manner as with various embodiments of a fluid delivery system described in the '771 patent incorporated by reference above.

The base portion includes an adhesive pad 222, an insertion mechanism 224, an activation mechanism 226, an engaging member 228 and a recess 230 sized and shaped to receive the fluid delivery cartridge 240. The base portion 220 is configured to be adhered to a patient's body with an adhesive layer disposed on the pad 222. The adhesive pad 222 can be relatively flat or shaped to mate with a particular body area. In some embodiments, the adhesive pad 222 can be an integral part of the base portion 220. For example, the adhesive pad 222 can include an adhesive deposited on an underside of the base portion 220. In other embodiments, the adhesive pad 222 can be a separate member coupled to the underside of the base portion 220. The adhesive can be any suitable adhesive that is non-toxic, biocompatible and releasable from human skin such as, for example, an acrylate based adhesive commonly used in bandages.

The insertion mechanism 224 is configured to insert a needle, catheter, cannula, infusion set, or other fluid delivery conduit into a patient for fluid delivery (also referred to herein as “fluid communicator”) when activated by the user via the activation mechanism 226. The insertion mechanism 224 can include one or more energy storage mechanisms such as a spring. For example, a variety of different types of springs can be used, such as, compression, extension, spring washers, Belleville, tapered, or other types of springs to achieve a desired output. The activation mechanism 226 can be in the form of a button that can be configured to activate the insertion mechanism 224 or the actuator (not shown). The insertion mechanism 224 can also be configured to place the fluid communicator (not shown) in fluid communication with a fluid source (not shown) such that it can communicate the fluid within the fluid source (not shown) to the patient as described herein. The insertion mechanism 224 can include a penetration cannula having one end configured to penetrate the patient's skin and another end configured to puncture the fluid source (not shown). The penetration cannula can define a lumen and be movably disposed within a lumen of the fluid communicator (not shown). For example, the insertion mechanism 224 can be configured to puncture the fluid source (not shown) upon activation to create a fluid path between the fluid source (not shown) and the fluid communicator (not shown).

The base portion 220 can also include an engaging member 228 which can be in the form of a protrusion, for example an arm, a latch, a tab or any other similar structure configured to selectively engage or mate with the fluid delivery cartridge 240. For example, as shown in FIG. 3, the engaging member 228 is disposed at a back surface of the insertion mechanism 224. In some embodiments, the engaging member 228 can be configured to provide an alignment feature and/or mechanism to ensure proper alignment of the base portion 220 with the fluid delivery cartridge 240. In other embodiments, the engaging member 228 can be configured as a locking mechanism to permanently couple the fluid delivery cartridge 240 with the base portion 220. In still other embodiments, the engaging member 228 can be configured to perform an activation function and/or any other function or a combination of functions.

As described herein, the recess 230 is configured to have a size and shape to receive the fluid delivery cartridge 240. The recess 230 can include slots, notches, detents, grooves, any other alignment and/or coupling mechanism or combination thereof to reversibly or irreversibly couple the base portion 220 to the fluid delivery cartridge 240. In some embodiments, the recess 230 can be a standard size and shape such that different size (e.g. volume) fluid delivery cartridges 240 can be used with the same base portion 220. In some embodiments, the recess 230 can be configured to only work with particular sized fluid delivery cartridges 240.

As shown in FIG. 3, the fluid delivery cartridge 240 includes a top housing 242, a bottom housing 246, a fluid coupling mechanism 248, an actuator sub-assembly 250 and optionally, a system engagement mechanism 252. The top housing 242 and the bottom housing 246 can be mechanically coupled together by, for example, a snap fit connection, or bonded together with an adhesive, heat welding, or other known coupling methods to form an interior region. The fluid coupling mechanism 248, the actuator sub-assembly 250, and the system engagement mechanism 252 can each be disposed within the interior region defined by the top housing 242 and bottom housing 246.

In some embodiments, the top housing 242 can include a fluid level indicator 244 having a visualization window configured to allow visualization of the fluid level in the fluid source (not shown). The fluid level indicator 244 can also include, for example, markings to indicate a percentage of an initial volume of fluid that has been delivered, a volume of fluid that has been delivered, and/or a fluid level remaining in the reservoir. The fluid level indicator 244 can be integrally formed with the top housing 242 during a manufacturing process (e.g., molding, extrusion, stamping, etc.), etched on the housing, or manufactured separately and adhered on the housing (e.g., a sticker or decal). For example, the top housing 242 can be formed from a substantially transparent material and a sticker having a visualization window and an adhesive can be adhered to the outside or inside portion of the top housing 242 to form the fluid level indicator 244.

The top housing 242 and the bottom housing 246 can also define an opening 243 positioned to be adjacent at least a portion of the insertion mechanism 224 and or the fluid communicator (not shown) when the fluid delivery cartridge 240 and base portion 220 are coupled together. For example, the fluid coupling mechanism 248 can be disposed in the interior region of the fluid delivery cartridge 240 adjacent the opening 243 so that it can provide a fluid pathway between the fluid source (not shown) and the fluid communicator (not shown) housed within the insertion mechanism 224. The fluid coupling mechanism 248 can include tubing/cannulas such as, for example, metal or plastic tubing, coupling members such as, for example, a T-connector, U-connector, circular connector, and/or linear connector, a resealable and/or self-sealing septum, or any other lumen containing fluidic connector or combination thereof.

In some embodiments, the fluid coupling mechanism 248 can include a septum that can be formed from a flexible material such as rubber, plastic, polyurethane, polycarbonate, silicone or any other flexible material or combination thereof. In some embodiments, the septum couples the fluid coupling mechanism 248 with the fluid communicator (not shown) via piercing of a needle, tube, catheter or cannula of the fluid communicator (not shown) through the septum. In some embodiments, the fluid coupling mechanism 248 can additionally be configured to communicate a fluid into the fluid source (not shown) from an external fluid source (not shown) for example when the delivery device 200 is not pre-filled when supplied to the user. In some embodiments, the fluid coupling mechanism 248 can include a first septum configured to establish communication between the fluid source (not shown) and fluid communicator (not shown) and a second septum configured to establish fluid communication between an external fluid source (not shown) and the fluid source (not shown) disposed in the fluid delivery cartridge 240.

In some embodiments, the system engagement mechanism 252 can be disposed in the interior region of the fluid delivery cartridge 240 adjacent the opening 243 defined by the top housing 242 and the bottom housing 246. In some embodiments, the system engagement mechanism 252 can include a lever 253 formed from metal, plastic or any other rigid material that is free to be moved from a first position to a second position. For example, the lever 253 can be configured to rotate about a pivot mount and/or slide with respect to the fluid delivery cartridge 240. In some embodiments, the movement of the system engagement mechanism 252 can be configured to provide a visual indication to the user that the fluid delivery cartridge 240 is properly and completely coupled to the base portion 220. For example, the movement of the lever 253 can reveal a system engaged indicator (not shown) previously hidden by the system engagement mechanism 252. In some embodiments, the engaging member 228 of the base portion 220 can be configured to be inserted though the opening 243 and selectively engage the engagement mechanism 252 to move the lever 253 from a first position to a second position. In some embodiments, the system engagement mechanism 252 can also be configured to turn the system on, for example, by closing an electrical circuit of the electrochemical actuator (not shown).

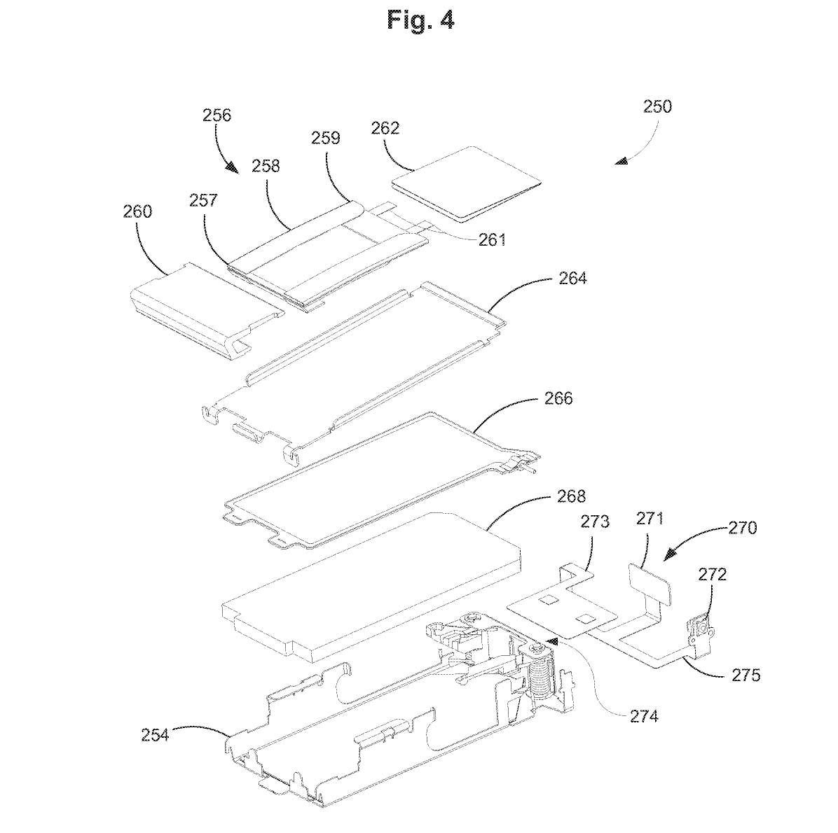

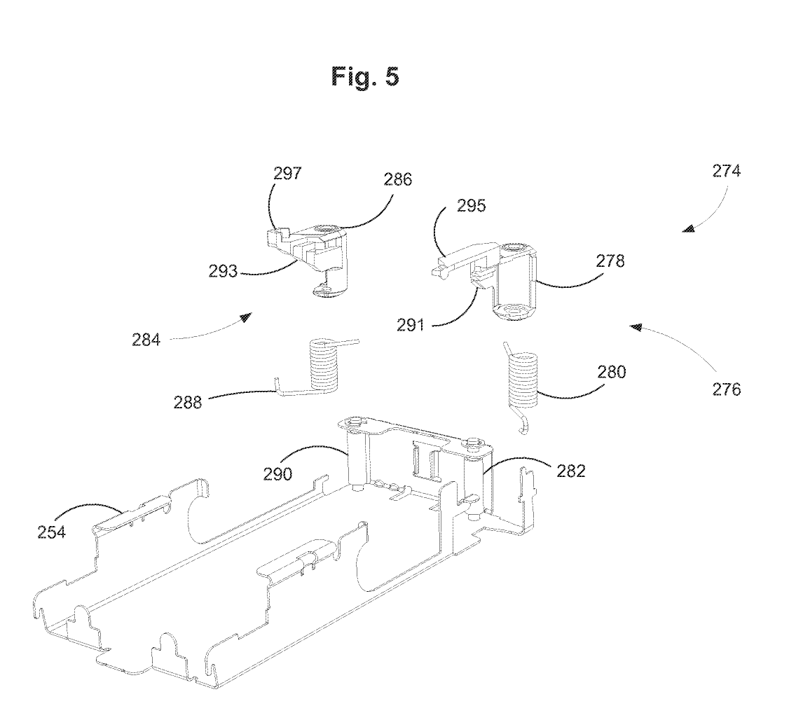

FIG. 4 illustrates an exploded view of the actuator sub-assembly 250 that can be included in the delivery device 200 of FIG. 2. The actuator sub-assembly 250 can include one or more actuators such as, for example, an electrochemical actuator and/or a mechanical actuator as described herein. In some embodiments, the actuator sub-assembly 250 includes a containment structure 254, an electrochemical actuator 256, a transfer structure 264, a fluid source 266, a current communicator 270 and (optionally) a mechanical actuator 274 (shown in further detail in FIG. 5).

The containment structure 254 can be configured to be a substantially rigid member that can be formed from rigid materials such as metals, plastics or a combination thereof. The containment structure 254 can be shaped and sized to have an interior region configured to at least partially house the components of the actuator sub-assembly 250. In some embodiments, the containment structure 254 can include mounting structures, for example notches, grooves, slots, indents, pins or a combination thereof that can serve as mounts, for example for mounting a constraining member 260 and/or the transfer structure 264. In some embodiments, the mounting structures can be pivots that, for example, allow pivotal coupling of the transfer structure 264 to the containment structure 254. In some embodiments, the containment structure 254 can be configured to contain all the forces generated by the electrochemical actuator 256 and/or the mechanical actuator 274 within the containment structure 254. For example, the containment structure 254 can be configured to ensure that all of the force generated by the actuators 256, 274 are transferred to the fluid source 266 (directly or indirectly) and not to the top housing 242 or bottom housing 246 (FIG. 3).

In some embodiments, the electrochemical actuator 256 can have a first end 257, a second end 259 and a medial portion 258. The first end 257 of the electrochemical actuator 256 is coupled to the containment structure 254 with the constraining member 260. In some embodiments, the constraining member 260 can include a clamp. The constraining member 260 can be configured so that when the electrochemical actuator 256 is actuated, the actuator bends in the medial portion 258 and produces a displacement of the unconstrained second end 259 towards the fluid source 266, for example, to exert a force on the fluid source 266. In some embodiments, the electrochemical actuator 256 bends uniformly from the first end 257 to the second end 259. Said another way, the medial portion 258 includes substantially all of the electrochemical actuator from the first end 257 to the second end 259. In some embodiments, the medial portion 258 only includes a portion of the electrochemical actuator between the first end 257 and the second end 259. The constraining member 260 can be formed from any suitable rigid material, for example, metals or plastics, or a combination thereof. Various other embodiments of constrained and unconstrained electrochemical actuator 256 are possible and can be found in the Electrochemical Actuator applications incorporated herein by reference in their entirety.

In some embodiments, a jacket 262 can be disposed on the second end 259 of the electrochemical actuator 256, such that it allows free motion of the second end 259 and, for example, serves to protect electrodes 261 on the second end 259 of the electrochemical actuator 256. The jacket 262 can include a substantially rigid member formed from insulating materials such as, for example, non-conducting metals, plastics, cardboard or a combination thereof. In some embodiments, the jacket 262 can further be configured to house a portion of the current communicator 270. For example, the jacket 262 can be configured to restrain a portion of the current communicator 270 such that it remains in current communication with electrodes 261 of the electrochemical actuator 256.

The transfer structure 264 is disposed between the electrochemical actuator 256 and the fluid source 266. The transfer structure 264 can include a rigid and substantially flat member that can be pivotally coupled to mounting structures on the containment structure 254 via pivots. In some embodiments, the transfer structure 264 can have a surface configured to engage the fluid source 266, such that a first force generated by the displacement of the second end 259 of the electrochemical actuator 256 is distributed by transfer structure 264 across a surface of the fluid source 266, for example to communicate fluid to the fluid communicator (not shown). In still other embodiments, the transfer structure 264 can also transfer a second force generated by the mechanical actuator 274 to the fluid source 264, in addition to the first force generated by electrochemical actuator 256.

The fluid source 266 can be provided to a user predisposed within the interior region of the actuator sub-assembly 250 or can be provided as a separate component that the user can insert into the actuator sub-assembly 250, for example through an opening (not shown). The fluid source 266 can be, for example, a fluid reservoir, bag or container, etc. that defines an interior volume that can contain a fluid to be injected into a patient. The fluid source 266 can also include a web portion (not shown) configured to be punctured by an insertion mechanism (not shown) to create a fluid channel between the fluid source 266 and a fluid communicator (not shown) configured to penetrate a patient's skin. In some embodiments, the fluid source 266 can be sized for example, with a length L of about 6 cm, a width W of about 3 cm, and a height H of about 0.2 cm to contain, for example, a total volume of 5 ml of fluid. In some embodiments, a compliant member 268 can be disposed between the fluid reservoir 266 and the containment structure 254. The compliant member 268 can be formed from substantially rigid but soft materials, for example foam pad, thick rubber, silicone, any other suitable material or combination thereof. In some embodiments, the compliant member 268 is a foam pad, that can be configured to prevent displacement of fluid source 266 in an axial direction perpendicular to the containment structure 254, for example to limit a compressive force delivered by the electrochemical actuator 256 and/or mechanical actuator 274 to the fluid source 266, thereby enabling complete communication of fluid contained in the fluid source 266 to the fluid communicator (not shown). In some embodiments, the compliant member 268 is configured to provide a structure against which a force-controlled deflection can be achieved to control the final motion of the electrochemical actuator 256 through the mechanical actuator 274 such that complete administration of the drug from the fluid reservoir is achieved. Said another way, the compliant member 268 allows the electrochemical actuator 256 to move the transfer structure 264 below the bottom of the mechanical actuator 274 so that the mechanical actuator 274 can completely open and empty the fluid source 266.

The current communicator 270 is configured to complete an electric circuit of the electrochemical actuator 256, for example, to activate the electrochemical actuator 256. In some embodiments, the current communicator 270 can include a single clip like member formed from a thin, flexible and conductive material such as, for example, a metal sheet that can include steel, aluminum, copper, a metal alloy, any other conductive material or combination thereof. In some embodiments, the current communicator 270 is a “flex-circuit” made of soft, flexible polyimide or polyester (or equivalent) material with conductive traces. The current communicator 270 can include a mounting portion 271 that can be rigidly mounted on a back face of the containment structure 254, for example by adhesive, screws, pins, rivets etc. on mounting structures such as holes, notches, slots on the containment structure 254. The current communicator 270 can further include a first arm 273 that can include a flat portion that may be used as the current conduit for the electrochemical actuator 256, and a second arm 275 that can have a switch 272 mounted on it. The switch 272 is configured to turn on the device 200 (i.e., completing the) when the engagement mechanism 252 is rotated over the switch 272. The rotary action compresses the switch 272 and completes the electrical circuit of the electrochemical actuator 256. In some embodiments, due to the flexibility of the current communicator 270, it will flex and move with the deforming electrochemical actuator 256 during movement from the first configuration t the second configuration.

Referring now to FIG. 5, the actuator sub-assembly 250 can include the mechanical actuator 274 configured to exert a force on the fluid source 266 during actuation of the electrochemical actuator 256. The mechanical actuator 274 can include, for example, a first spring based actuator 276 and a second spring based actuator 284.

In some embodiments, the first spring base actuator 276 can include a first rotary compression member 278 that may be formed from a rigid material such as, for example, plastics, metals, any other suitable material or combination thereof. The first rotary compression member 278 can be coupled to a torsion spring 280 and configured such that when the first torsion spring 280 is released from its compressed (or “twisted”) configuration, the first rotary compression member 278 applies a first compressive force to the transfer structure (not shown) as described in further detail below. The first rotary compression member 278 can be mounted on a first mounting pin 282 that can be disposed on mounts, for example, holes, notches, indents, slots, pivots, etc. at one end of the containment structure 254 proximate to the second end 259 of the electrochemical actuator (not shown).

In some embodiments, the second spring based actuator 284 can also include a second rotary compression member 286 that may be formed from a rigid material such as, for example, plastics, metals, any other suitable material or combination thereof. Similar to the first rotary compression member 278, the second rotary compression member 286 can be coupled to a second torsion spring 288, disposed and configured similar to the first spring based actuator 280. The second rotary compression member 286 can be mounted on a second mounting pin 290, that can be disposed on mounts at an end of the containment structure 254 proximate to the second end 259 of the electrochemical actuator (not shown).

In some embodiments, the first rotary compression member 278 and the second rotary compression member 286 can be configured to convert the rotational motion of the rotary compression members 278, 286 to an axial compressive force on the transfer structure (not shown), which is further transferred to the fluid source (not shown). For example, the rotary compression members 278, 286 can include compression structures 291, 293 having an edge or a surface shaped to engage and apply a compressive force on the transfer structure (not shown). The shaped surface can be, for example, tapered, curved (e.g. concave or convex) or can include asymmetric or symmetric gradations (e.g. steps with chamfered or filleted edges) or a combination thereof. In some embodiments, the compression structures 291, 293 are steps with filleted edges, configured to apply a second axial compressive force on the transfer structure in conjunction with a first compressive force applied on the transfer structure (not shown) by the electrochemical actuator (not shown).

In some embodiments, the first rotary compression member 278 can also include a fluid level indicator 295 that can include, for example, a dial like structure, an arm, a strut or a combination thereof. The fluid level indicator 295 can be a separate member mounted on the first rotary compression member 278 or maybe an integral part of the first rotary compression member 278. For example, the fluid level indicator 295 can be integrally formed with the first rotary compression member 278 in a single molding, stamping, milling, any other suitable process or combination thereof. In some embodiments, the fluid level indicator 295 is configured to be viewed through a visualization window (not shown). In this configuration, rotational displacement of the fluid level indicator 295 can be correlated to markings on the visualization window (not shown) such that the fluid level indicator 295 indicates a percentage, or a volume of fluid that has been communicated through the fluid communicator (not shown), or a fluid level remaining in the fluid source.

In some embodiments, the second rotary compression member 286 can include a system off structure 297 configured to interact with the current communicator 270 (FIG. 4) at the end of a delivery cycle such that the current communicator 270 is disengaged from electrical communication with the electrochemical actuator 256 (FIG. 4). This can, for example, turn the electrochemical actuator 256 off.

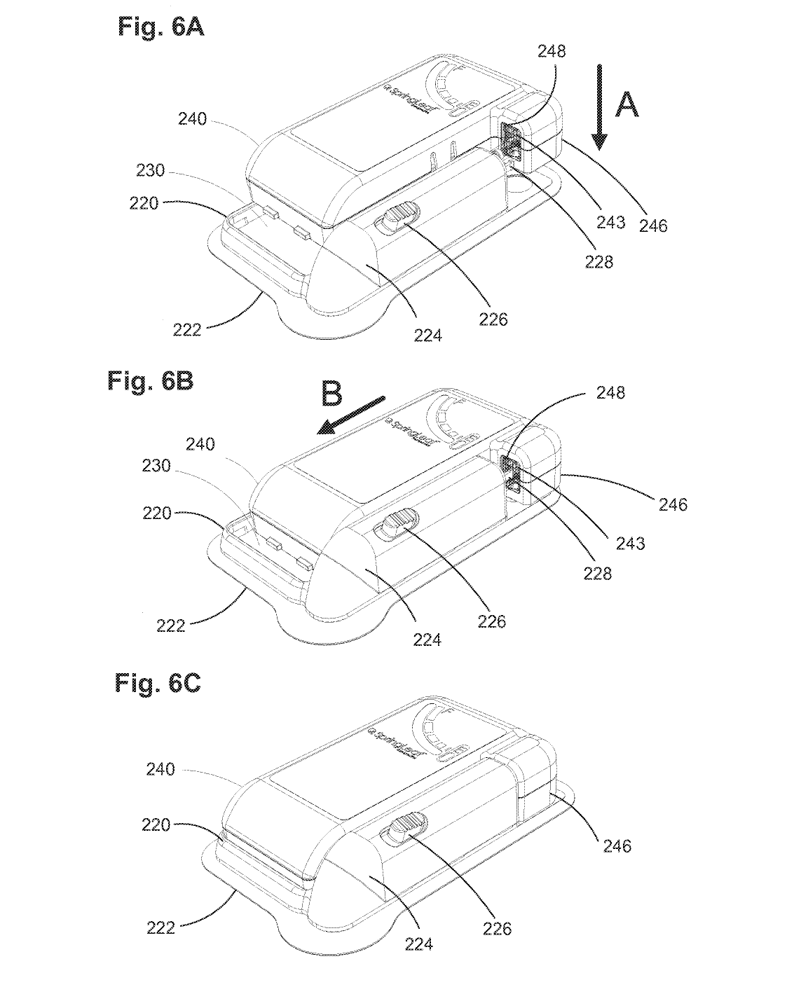

As described above, the base portion 220 and the fluid delivery cartridge 240 can be pre-assembled and permanently coupled together, removably coupled to each other, and/or separate components that are assembled by the user. For example, referring now to FIGS. 6A-6C, the base portion 220 and the fluid delivery cartridge 240 are separate components that are assembled prior to use by the user. In some embodiments, it may be desirable to have separate components so that the base portion 220 and the fluid delivery cartridge 240 can be packaged separately and stored in different environments (e.g., inert atmosphere, vacuum sealed, temperature or humidity controlled, etc.).

As shown in FIG. 6A, the base portion 220 and fluid delivery cartridge 240 are initially separated and brought into contact by the user in a direction as indicated by arrow A until the bottom housing 246 of the fluid delivery cartridge 240 contacts the recess 230 of the base portion. In some embodiments, the fluid delivery cartridge 240 is positioned in a plane substantially parallel to a plane defined by the base portion 220 such that the fluid delivery cartridge 240 is above the base portion and slightly offset from the recess 230 as shown in FIG. 6A. A first force is applied to fluid delivery cartridge 240 to push it downward in a direction indicated by arrow A until a bottom surface of the bottom housing 246 is flush with a top surface of the recess 230, and the engaging member 228 is aligned with and adjacent to the opening 243 as shown in FIG. 6B. In some embodiments, a front portion of the fluid delivery cartridge 240 can be angled downward and placed in contact with the base portion 220 and a back portion of the fluid delivery cartridge 240 can be rotated in a downward direction until the bottom surface of the bottom housing 246 is flush with the top surface of the recess 230. Once the fluid delivery cartridge 240 is properly positioned and aligned with the base portion 220, a second force is applied to the fluid delivery cartridge 240 in a direction indicated by arrow B such that the fluid delivery cartridge 240 slides in the recess until it reaches the fully coupled configuration as shown in FIG. 6C. As described herein, the fluid delivery cartridge 240 and/or the base portion 220 can include slots, notches detent grooves, any other coupling mechanism or combination thereof configured to permanently or releasably couple the fluid delivery cartridge 240 with the base portion 220.

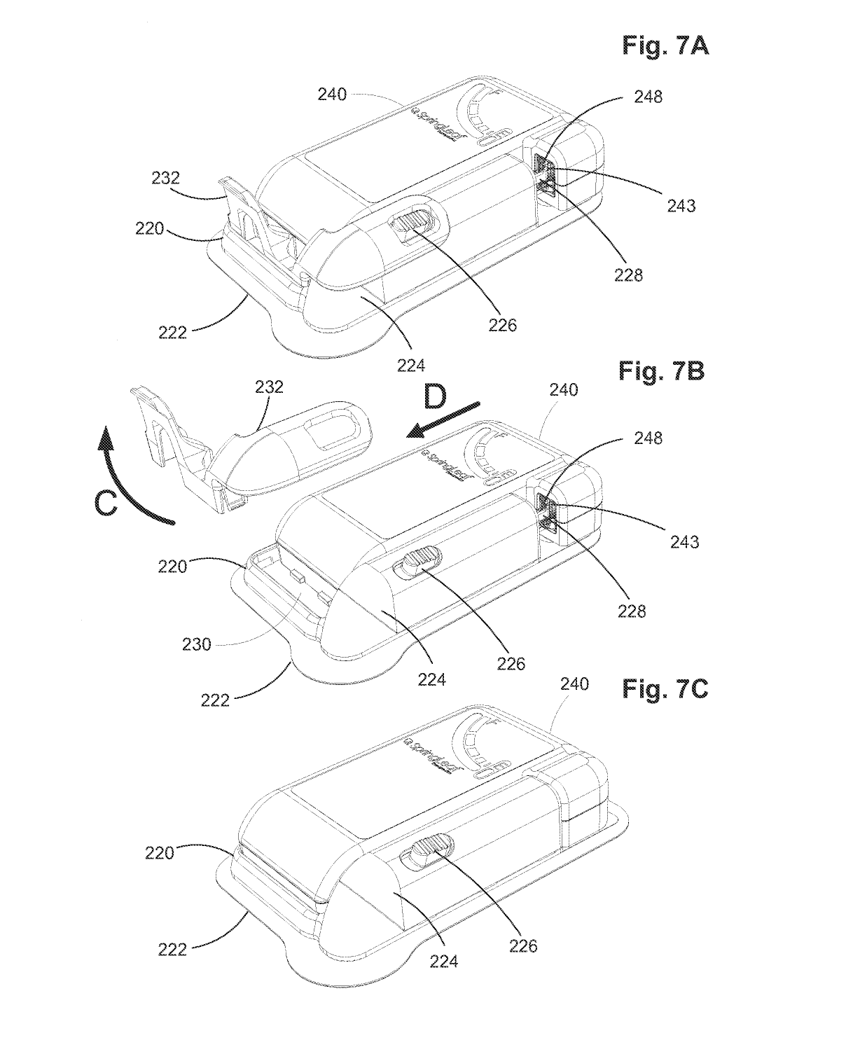

In some embodiments, the base portion 220 and the fluid delivery cartridge 240 are at least partially pre-assembled and then final assembly is performed by the user prior to use. For example, referring now to FIGS. 7A-7C, the fluid delivery cartridge 240 is attached to the base portion 220 and a safety clip 232 is disposed between the base portion 220 and the fluid delivery cartridge 240. The safely clip 232 is configured to prevent inadvertent coupling of the fluid delivery cartridge 240 to the base portion 220. In some embodiments, the safety clip 232 is a rigid member formed from a material such as plastic, metal or a combination thereof. In some embodiments, the safety clip 232 can be shaped and sized to fit at least partially in the recess 230 between the base portion 220 and the fluid delivery cartridge 240. In some embodiments, the safety clip 232 can include a coupling mechanism for example notches, grooves, slots, detents, any other coupling mechanism or a combination thereof configured to reversibly couple the safety clips to the base portion 220 or the fluid delivery cartridge 240. The safety clip 232 can be configured to be disposed over at least a portion of the insertion mechanism 224. In some embodiments, the safely clip 232 can include an opening configured to at least partially surround the activation mechanism 226 to prevent accidental depression and/or other movement of the activation mechanism 226. As shown in FIG. 7A, the safety clip 232 is positioned between the fluid delivery cartridge 240 and the base portion 220, and disposed partially over the insertion mechanism 224. The engaging member 228 is also aligned with and adjacent to the opening 243. When ready for use, the user can remove the safety clip 232 as indicated by the arrow C in FIG. 7B, and slide the fluid delivery cartridge 240 in a direction indicated by arrow D, such that the fluid delivery cartridge 240 slides in the recess 230 until it reaches the fully coupled configuration as shown in FIG. 7C. The assembled device 200 can have a smooth shape that is relatively free from sharp edges and may be concealed under clothing.

In some embodiments, in the fully coupled configuration as shown in FIGS. 6C and 7C, the engaging member 228 protrudes into the fluid delivery cartridge 240 through the opening 243. In some embodiments, the engaging member 228 can be configured to provide an alignment mechanism for coupling the base portion 220 with the fluid delivery cartridge 240. In other embodiments, the engaging member 228 can provide a locking mechanism. In still other embodiments, the engaging member 228 performs an activation function. For example, the engaging member 228 can disengage a locking mechanism such as, for example, the engagement mechanism 252 shown in FIG. 3. This can, for example, bring the system in an active state ready and for use. Once in the active state, the user can move the activation mechanism 226 from an off to an on position thereby activating the insertion mechanism 224 and/or turning an electrochemical and/or mechanical actuator (not shown) on. In some embodiments, the activation mechanism 226 can be placed in the ON position once applied to the patient, and after visual inspection of the insertion site, the patient can activate the device 200 by coupling the engaging member 228 with the engagement mechanism 252. In other embodiments, the engaging member 226 can be configured to close an electrical circuit to turn the electrochemical actuator (not shown) on, subsequent to the assembly of the delivery device 200. The activation mechanism 226 can then be configured to activate the insertion mechanism 224 only. In still further embodiments, the engaging member 228 can be configured to activate the insertion mechanism 224 on assembly of the delivery device 200 and the activation mechanism 226 can then be used, for example to turn the electrochemical and/or mechanical actuator on.

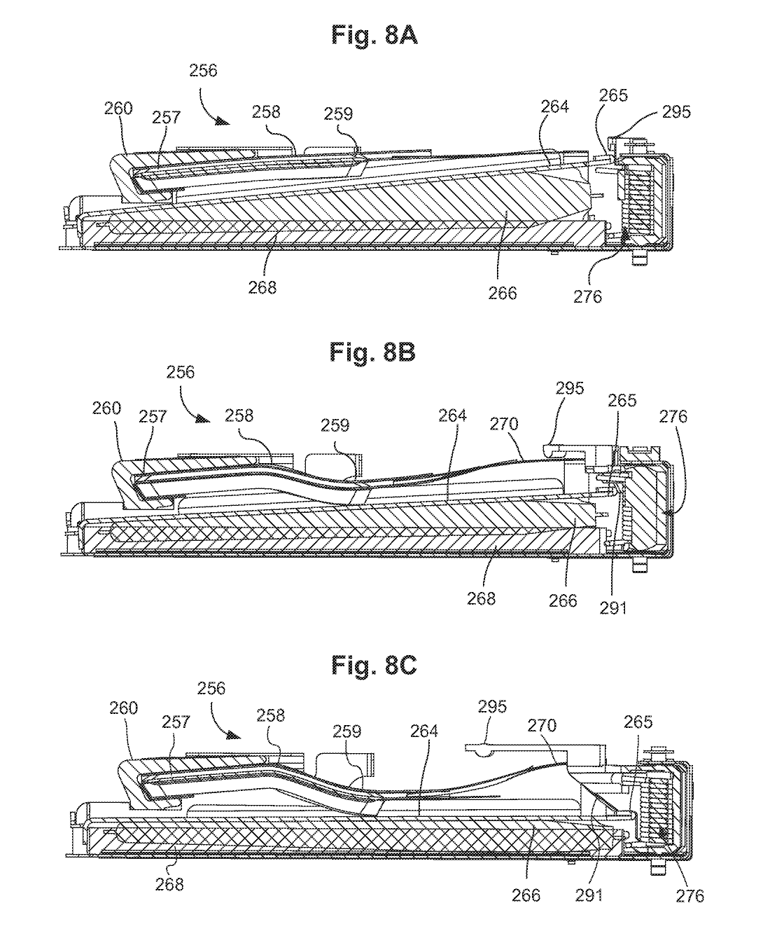

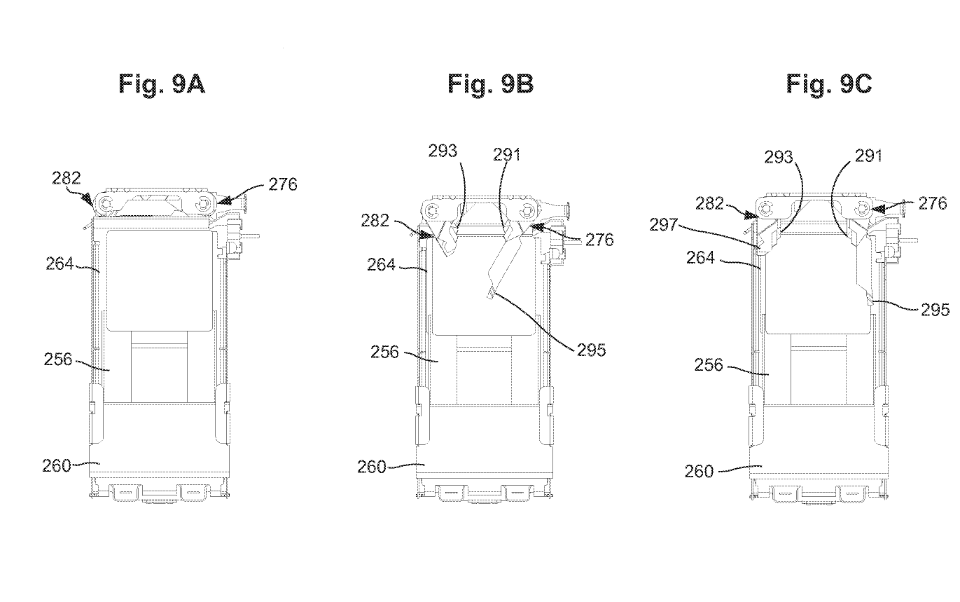

Referring now to FIGS. 8A-8C and FIGS. 9A-9C, an actuator sub-assembly 250 of FIG. 3 of the fluid delivery device 200 of FIG. 2 is shown in a first (FIGS. 8A and 9A), a second (FIGS. 8B and 9B) and a third (FIGS. 8C and 9C) configuration. As described herein, the actuator sub-assembly 250 includes an electrochemical actuator 256, a transfer structure 264, a fluid source 266, a first spring based actuator 276 and a second spring based actuator 282.

In the first configuration, the first end 257 of the electrochemical actuator 256 is constrained by a constraining member 258, and the spring based actuators 276, 282 are in a first position in a fully compressed state. In this configuration, the spring based actuators 276, 282 substantially resemble closed doors (e.g., spring-loaded saloon doors). In some embodiments, movement of the spring based actuators 276, 282 is prevented by an edge 265 of the transfer structure 264 in the first configuration.

Once activated (e.g., by closing an electric circuit as described herein), the electrochemical actuator 256, with the first end 257 constrained by constraining member 260, begins bending in the medial portion 258 and produce a displacement of the second end 259 towards the transfer structure 264 thereby exerting a force on the fluid source 266. The transfer structure 264, disposed between the electrochemical actuator 256 and fluid source 266, can be configured to distribute the force exerted by the electrochemical actuator 256 across a surface of the fluid source 266. As the transfer structure 264 is moved by the deflection of the electrochemical actuator 256, the edge 265 of the transfer structure 264 in contact with spring based actuators 276, 282 moves in a downward direction (e.g., the edge 265 is placed in contact with the compression structures 291, 293) allowing the spring based actuators 276, 282 to begin actuation. This allows the spring based actuators 276, 282 to move from a first configuration to a second configuration as shown in FIGS. 8B and 9B such that the compression structures 291, 293 exert a second axial compressive force on the transfer structure 264. In some embodiments, the combination of the first force of the electrochemical actuator 256 and the second force of the spring based actuators 276, 282 collectively urge the transfer structure 264 towards the fluid source 266 such that the fluid, for example a drug within the fluid source 266 is communicated through the fluid communicator (not shown).

As described above and as best shown in FIGS. 9B and 9C, the first spring based actuator 276 can include a fluid level indicator 295 that can be visualized by the user as the first spring based actuator 276 moves from the first configuration to the second and third configurations. The fluid level indicator 295 can be configured, for example to be viewed through a visualization window 244 of a top housing 242 of the delivery device 200. In some embodiments, the fluid level indicator 295 can indicate the percent of initial fluid or fluid volume communicated to the fluid communicator (not shown) or the fluid volume remaining in the fluid source 266.

The delivery cycle of delivery device 200 ends when substantially all of the fluid is communicated from fluid source 266, through the fluid communicator (not shown), and to the user. For example, as illustrated in FIGS. 8C and 9C, the electrochemical actuator 256 and the spring based actuators 276, 282 are fully actuated such the transfer structure 264 is completely depressed and the fluid source 266 is substantially empty (e.g., at least most of the fluid in fluid source 266 has been communicated to the fluid communicator). In some embodiments, the second spring based actuator 282, can include a system off structure 297 (shown best in FIG. 9C) configured to interact with the current communicator 270 in the third configuration and disengage the current communicator 270 from electrochemical actuator 256, thereby opening the electric circuit of the electrochemical actuator 256 and turning the delivery device 200 off. Once the delivery cycle is completed, the user can remove the delivery device 200 from their skin and dispose of the device. In some embodiments, the user can remove only the fluid delivery cartridge 240 and replace it with a new fluid delivery cartridge 240 to deliver a second dose of the drug being delivered or a completely new drug.

A delivery device as described herein may be used to deliver a variety of drugs according to one or more release profiles. For example, the drug may be delivered according to a relatively uniform flow rate, a varied flow rate, a pre-programmed flow rate, a modulated flow rate, in response to conditions sensed by the device, in response to a request or other input from a user or other external source, or combinations thereof. Thus, embodiments of the delivery device may be used to deliver drugs having a short half-life, drugs having a narrow therapeutic window, drugs delivered via on-demand dosing, normally-injected compounds for which other delivery modes such as continuous delivery are desired, drugs requiring titration and precise control, and drugs whose therapeutic effectiveness is improved through modulation delivery or delivery at a non-uniform flow rate. These drugs may already have appropriate existing injectable formulations.

For example, the delivery devices may be useful in a wide variety of therapies. Representative examples include, but are not limited to, opioid narcotics such as fentanyl, remifentanyl, sufentanil, morphine, hydromorphone, oxycodone and salts thereof or other opioids or non-opioids for post-operative pain or for chronic and breakthrough pain; NonSteroidal Antinflamatories (NSAIDs) such as diclofenac, naproxen, ibuprofin, and celecoxib; local anesthetics such as lidocaine, tetracaine, and bupivicaine; dopamine antagonists such as apomorphine, rotigotine, and ropinerole; drugs used for the treatment and/or prevention of allergies such as antihistamines, antileukotrienes, anticholinergics, and immunotherapeutic agents; antispastics such as tizanidine and baclofin; insulin delivery for Type 1 or Type 2 diabetes; leutenizing hormone releasing hormone (LHRH) or follicle stimulating hormone (FSH) for infertility; plasma-derived or recombinant immune globulin or its constituents for the treatment of immunodeficiency (including primary immunodeficiency), autoimmune disorders, neurological and neurodegenerative disorders (including Alzheimer's Disease), and inflammatory diseases; apomorphine or other dopamine agonists for Parkinson's disease; interferon A for chronic hepatitis B, chronic hepatitis C, solid or hematologic malignancies; antibodies for the treatment of cancer; octreotide for acromegaly; ketamine for pain, refractory depression, or neuropathic pain; heparin for post-surgical blood thinning; corticosteroid (e.g., prednisone, hydrocortisone, dexamethasone) for treatment of MS; vitamins such as niacin; Selegiline; and rasagiline. Essentially any peptide, protein, biologic, or oligonucleotide, among others, that is normally delivered by subcutaneous, intramuscular, or intravenous injection or other parenteral routes, may be delivered using embodiments of the devices described herein. In some embodiments, the delivery device can be used to administer a drug combination of two or more different drugs using a single or multiple delivery port and being able to deliver the agents at a fixed ratio or by means enabling the delivery of each agent to be independently modulated. For example, two or more drugs can be administered simultaneously or serially, or a combination (e.g. overlapping) thereof.

In some embodiments, the delivery device may be used to administer ketamine for the treatment of refractory depression or other mood disorders. In some embodiments, ketamine may include either the racemate, single enantiomer (R/S), or the metabolite (wherein S-norketamine may be active). In some embodiments, the delivery devices described herein may be used for administration of Interferon A for the treatment of hepatitis C. In one embodiment, a several hour infusion patch is worn during the day or overnight three times per week, or a continuous delivery system is worn 24 hours per day. Such a delivery device may advantageously replace bolus injection with a slow infusion, reducing side effects and allowing the patient to tolerate higher doses. In other Interferon A therapies, the delivery device may also be used in the treatment of malignant melanoma, renal cell carcinoma, hairy cell leukemia, chronic hepatitis B, condylomata acuminata, follicular (non-Hodgkin's lymphoma, and AIDS-related Kaposi's sarcoma.

In some embodiments, a delivery device as described herein may be used for administration of apomorphine or other dopamine agonists in the treatment of Parkinson's Disease (“PD”). Currently, a bolus subcutaneous injection of apomorphine may be used to quickly jolt a PD patient out of an “off” state. However, apomorphine has a relatively short half-life and relatively severe side effects, limiting its use. The delivery devices described herein may provide continuous delivery and may dramatically reduce side effects associated with both apomorphine and dopamine fluctuation. In some embodiments, a delivery device as described herein can provide continuous delivery of apomorphine or other dopamine agonist, with, optionally, an adjustable baseline and/or a bolus button for treating an “off” state in the patient. Advantageously, this method of treatment may provide improved dopaminergic levels in the body, such as fewer dyskinetic events, fewer “off” states, less total time in “off” states, less cycling between “on” and “off” states, and reduced need for levodopa; quick recovery from “off” state if it occurs; and reduced or eliminated nausea/vomiting side effect of apomorphine, resulting from slow steady infusion rather than bolus dosing.

In some embodiments, a delivery device as described herein may be used for administration of an analgesic, such as morphine, hydromorphone, fentanyl or other opioids, in the treatment of pain. Advantageously, the delivery device may provide improved comfort in a less cumbersome and/or less invasive technique, such as for post-operative pain management. Particularly, the delivery device may be configured for patient-controlled analgesia.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. For example, although some embodiments were not described as including an insertion mechanism, an activation mechanism, electrical circuitry, etc., it should be understood that those embodiments of a delivery device can include any of the features, components and/or functions descried herein for other embodiments. In addition, the specific configurations of the various components can also be varied. For example, the size and specific shape of the various components can be different than the embodiments shown, while still providing the functions as described herein.

1. An apparatus, comprising:
a reservoir configured to contain a fluid;
a fluid communicator configured to be placed in fluid communication with the reservoir;
a first actuator having a first end, a second end, and a medial portion between the first end and the second end, the first actuator being disposed with the first end constrained, the second end unconstrained, and configured so that when actuated, the first actuator bends in the medial portion and produces a displacement of the second end and exerts a first force on the reservoir;
a transfer structure disposed between the first actuator and the reservoir, the transfer structure having a surface configured to engage the reservoir such that the first force exerted by the first actuator is distributed by the transfer structure across a surface of the reservoir engaged by the transfer structure; and
a second actuator having a first configuration and a second configuration, the second actuator being disposed and oriented so that when the second end of the first actuator is displaced toward the fluid reservoir, the second actuator moves from its first configuration toward its second configuration and exerts a second force on the transfer structure,
the combination of the first force and the second force collectively configured to urge the transfer structure toward the reservoir such that fluid within the reservoir is communicated through the fluid communicator.

2. The apparatus of claim 1, wherein the first actuator is an electrochemical actuator.

3. The apparatus of claim 1, wherein the second actuator is a mechanical spring-based actuator.

4. The apparatus of claim 1, wherein the second actuator includes a rotary compression member and a spring, the spring configured to rotate the rotary compression member from the first configuration to the second configuration.

5. The apparatus of claim 1, wherein the spring is a torsion spring.

6. The apparatus of claim 4, wherein the rotary compression member has a tapered surface, the tapered surface configured to exert the second force on the transfer structure when the second actuator moves from the first configuration to the second configuration.

7. The apparatus of claim 4, wherein the rotary compression member has a stepped surface, the stepped surface configured to exert the second force on the transfer structure when the second actuator moves from its first configuration to its second configuration.

8. The apparatus of claim 1, wherein the second actuator includes a first rotary compression member and a second rotary compression member, the first rotary compression member and the second rotary compression member each including a spring configured to rotate the first and second rotary compression members from the first configuration to the second configuration.

9. The apparatus of claim 1, wherein the second actuator includes a system off structure, the system off structure configured to turn the system off after substantially all of the fluid contained in the reservoir is communicated to the fluid communicator.

10. An apparatus, comprising:
a housing configured to be removably coupled to a user;
a reservoir configured to contain a fluid and disposed within the housing; and
an actuator having a first end, a second end, and a medial portion between the first end and the second end, the actuator being disposed with the first end constrained, the second end unconstrained, and configured so that when actuated, the actuator bends in the medial portion and produces a displacement of the second end and exerts a first force on the reservoir; and
a containment structure coupled to the first end of the actuator and configured so that when the second end of the actuator is displaced toward the fluid reservoir, substantially all of the first force generated by the displacement of the second end of the actuator is transferred to the reservoir and not to the housing.

11. The apparatus of claim 10, further comprising:
a constraining member disposed at the first end of the actuator and configured to mechanically couple the actuator to the containment structure.

12. The apparatus of claim 11, wherein the constraining member is a clamp.

13. The apparatus of claim 10, further comprising:
a transfer structure disposed between the actuator and the reservoir, the transfer structure having a surface configured to engage the reservoir such that the first force exerted by the first actuator is distributed by the transfer structure across a surface of the reservoir engaged by the transfer structure.

14. The apparatus of claim 10, wherein the actuator is a first actuator, the apparatus further comprising:
a second actuator having a first configuration and a second configuration, the second actuator being disposed and oriented so that when the second end of the first actuator is displaced toward the fluid reservoir, the second actuator moves from its first configuration to its second configuration and exerts a second force on the transfer structure.

15. The apparatus of claim 14, wherein the a containment structure is configured so that when the second actuator moves from its first configuration to its second configuration and exerts the second force on the transfer structure, substantially all of the second force generated by the second actuator is transferred to the reservoir and not to the housing.

16. An apparatus, comprising:
a housing configured to be removably coupled to a user and having a visualization window;
a reservoir configured to contain a fluid;
a first actuator having a first end, a second end, and a medial portion between the first end and the second end, the first actuator being disposed with the first end constrained, the second end unconstrained, and configured so that when actuated, the first actuator bends in the medial portion and produces a displacement of the second end and exerts a first force on the reservoir;
a transfer structure disposed between the first actuator and the reservoir, the transfer structure having a surface configured to engage the reservoir such that the first force exerted by the first actuator is distributed by the transfer structure across a surface of the reservoir engaged by the transfer structure; and
a second actuator having a first configuration and a second configuration, the second actuator being disposed and oriented so that when the second end of the first actuator is displaced toward the fluid reservoir, the second actuator moves from its first configuration toward its second configuration and exerts a second force on the transfer structure,
the second actuator including a fluid level indicator that is visible through the visualization window as the second actuator moves from the first configuration to the second configuration.

17. The apparatus of claim 16, further comprising:
a fluid communicator configured to be placed in fluid communication with the reservoir.

18. The apparatus of claim 17, wherein the combination of the first force and the second force collectively urge the transfer structure toward the reservoir such that fluid within the reservoir is communicated through the fluid communicator.

19. The apparatus of claim 18, wherein the visualization window includes markings to indicate a percentage of an initial volume of fluid that has been communicated through the fluid communicator.

20. The apparatus of claim 18, wherein the visualization window includes markings to indicate a volume of fluid that has been communicated through the fluid communicator.

21. The apparatus of claim 18, wherein the visualization window includes markings to indicate a fluid level remaining in the reservoir.
